(19) United States
(12) Patent Application Publication
(10) Pub. No.: US 2009/0171232 A1
(43) Pub. Date: Jul. 2, 2009

(54) Drowsiness detection system

(76) Inventors: Wei-Chih HU, Ping Zhen City (TW); Liang-Yu Shyu, Ban Qiao City (TW); Pai-Yuan Tsai, Tai Ping City (TW); Hsin-Chang Chen, Ban Qiao City (TW)

Correspondence Address: SINORICA, LLC, 2275 Research Blvd., Suite 500, ROCKVILLE, MD, 20850, US

(21) Appl. No.: 12/122,798

(22) Filed: May 19, 2008

(30) Foreign Application Priority Data
Dec. 28, 2007 (TW) 096150929

(51) Int. Cl. A61B 5/0476

(52) U.S. Cl. 600/545; 600/544

(57) ABSTRACT

A drowsiness detection system is disclosed. The way of detection is by an EEG detection circuit to detect brain waves of a human brain for getting an EEG signal. The EEG signal is sent to a micro-controller circuit to generate a control signal. In accordance with the control signal, a processing circuit processes the EEG signal so as to learn the drowsiness state of the user in time.

### BACKGROUND OF THE INVENTION

The present invention relates to a detection system, especially to a drowsiness detection system.

In factors that lead to traffic accidents, driver's fatigue is one of the most important factors. Sleepiness caused by a plurality of factors such as long distance drive on highways, a feeling of boredom and monotony that lead to impatience and fatigue, or after-meal drowsiness. The sleepiness may cause impairment of alertness of the driver, reacting slowly to driving situations, attention deficit so that it is very dangerous to drive under such condition and may result in serious injury or fatal accident.

Thus there is a need to have a safe, high-reliable, in-time monitoring detection to detect driver's drowsiness, warn the driver to avoid accidents. There are several ways available now to detect drowsiness of the driver. By direct image capture or electrooculographic potential (EOG), eye-blinking frequency is observed.

When there is a change in Eyelid movements (EM)—reduced blinking rate, the driver may become drowsy. In physiological measurements, parameters such as electrocardiogram (ECG), blood pressure, respiration and electroencephalogram (EEG) were recorded for evaluation of drowsiness. When the driver is tired or fatigue, some specific signals show in EEG and the drowsiness is detected thereby. However, devices required by above method are quire large and inconvenient to carry with. Moreover, EEG signal provides a lot of information of driver's alertness and an analysis of driver's alertness is mostly done by off-line processing of a computer. Thus it lacks in-time monitoring function. Thus there is a need to provide a novel drowsiness detection system that retrieves signals of different frequencies from stationary wavelet through a non-invasive EEG Then characteristic signals are found from the separated signals of different frequencies and then further are characterized. Next the signals are classified and identified by a neural network. When the driver is tired, the system automatically detects the driver's status and warn the driver just in time so as to prevent above problems.

### SUMMARY OF THE INVENTION

Therefore it is a primary object of the present invention to provide a drowsiness detection system and a method thereof that detect the driver's fatigability in time by a processing circuit that processes an EEG(electroencephalogram) signal.

It is another object of the present invention to provide a drowsiness detection system and a method thereof that detect the drowsiness of bodies by a neural network.

In order to achieve above objects, the present invention includes an EEG detection circuit, a micro-control circuit and a processing circuit. The way to detect drowsiness of the driver is by the EEG detection circuit to get an EEG signal of a human brain. The micro-control circuit receives the EEG signal and generates a control signal that is sent to the processing circuit. In accordance with the control signal, the processes and analyzes the EEG signal so as to learn the fatigability of the person.

Moreover, the processing circuit includes a conversion unit, a processing unit and a recognition unit. The conversion unit receives and converts the EEG signal into a conversion signal while the processing unit receives and processes the conversion signal to generate a processing signal that is sent to the recognition unit for generating a detection result related to the drowsiness of the body. The detection result is sent back to the micro-control circuit for output of the detection result.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a block diagram of an embodiment according to the present invention;

FIG. 2 is a block diagram of an EEG detection circuit of an embodiment according to the present invention;

FIG. 3 shows disposition of electrodes of the embodiment according to the present invention;

FIG. 4 is a block diagram of a processing circuit of the embodiment according to the present invention;

FIG. 5 is a flow chart of the processing circuit of the embodiment according to the present invention.

### DETAILED DESCRIPTION OF THE PREFERRED EMBODIMENT

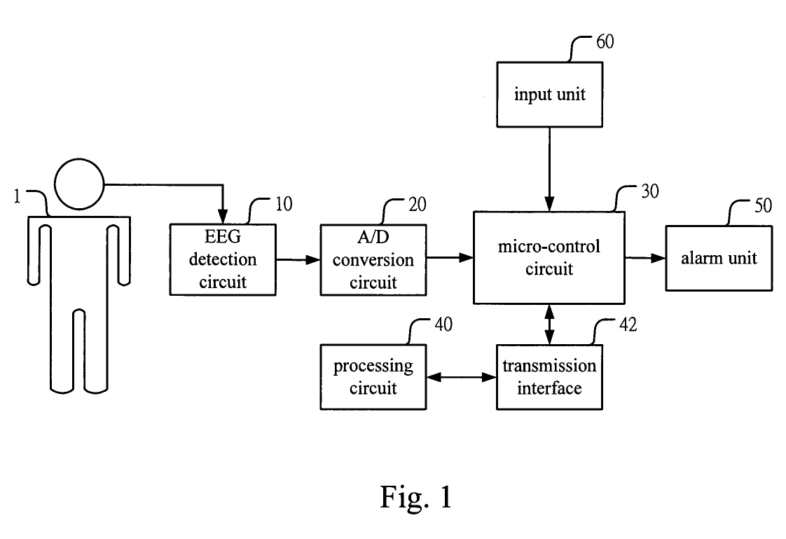

Refer to FIG. 1, a drowsiness detection system of the present invention consists of an EEG detection circuit 10, an analog-to-digital(A/D) conversion circuit 20, a micro-control circuit 30 and a processing circuit 40. The EEG detection circuit 10 is to detect electrical activity of a user's brain to generate an EEG signal. The micro-control circuit 30 receives the EEG signal to generate a control signal. According to the control signal, the processing circuit 40 processes and analyzes the EEG signal so as to learn the fatigability of the person 1. Moreover, the system of the present invention further includes an alarm unit 50 coupled to the micro-control circuit 30. When the processing and analysis result of the processing circuit 40 shows that the person is tired, the alarm unit 50 sends a warning signal so as to inform the driver that he is in fatigue state and needs to take a rest. The alarm unit 50 can be a light emitting device such as a light emitting diode (LED) or a light bulb. Or it can be an audio device such as a speaker or a buzzer that sends out an alarming sound to the user.

Furthermore, a transmission interface 42 is disposed between the micro-control circuit 30 and the processing circuit 40 so as to receive 6-channel EEG signal as well as various commands from the micro-control circuit 30 and send some output results to the micro-control circuit 30 for being displayed. Data transmission between the micro-control circuit 30 and the processing circuit 40 takes place in a parallel way for increasing data transmission speed. The transmission interface 42 is an Enhanced Host-Port Interface (EHPI).

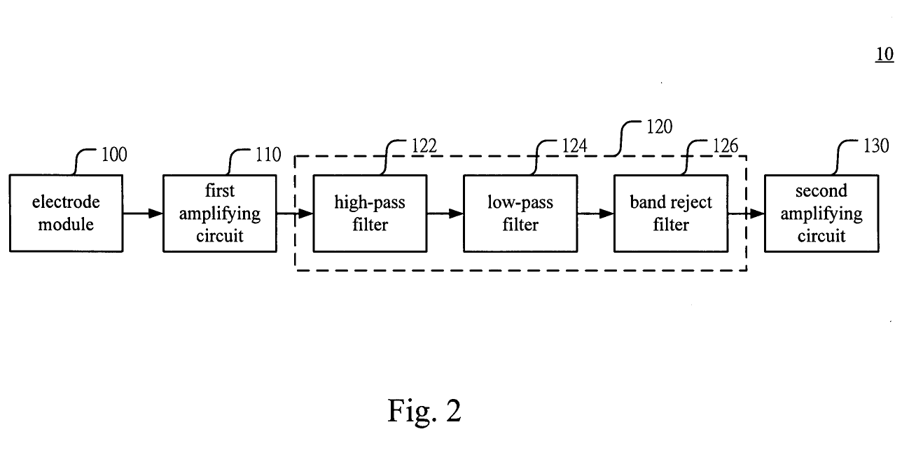

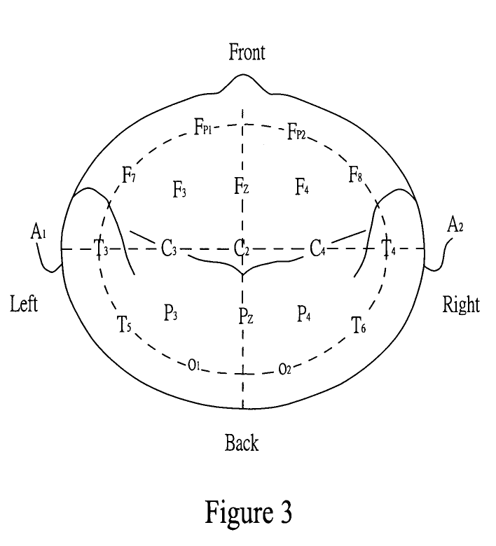

Refer to FIG. 2, the EEG detection circuit 10 is composed of an electrode module 100, a first amplifying circuit 110, a filter circuit 120 and a second amplifying circuit 130. The electrode module 100 attaches on the head to detect the EEG signal generated from the person's 1 brain. The electrode module 100 includes six electrodes that are disposed according to convenience of use and certain area with higher drowsiness reaction. The electrodes can be arranged on a hat so that the driver can use this system by wearing the hat. When the driver is tired, a α wave appears in the EEG signal while the detection of the a wave is more obvious at parietal lobe and occipital lobe. Thus the electrodes are arranged on the FP1, FP2, T5, T6, O1 and O2, as shown in FIG. 3. The measurement is by unipolar recording so that a reference electrode is required. Thus the point A2 works as reference of all electrodes while the grounding is on the A1 position.

The first amplifier circuit 110 is an instrumentation amplifier. Because the brain wave signal (EEG signal) is quite weak and instable, the first amplifier circuit 110 receives the EEG signal detected by the electrode module 100 for amplifying weak psychological (brain wave) signal while the filter circuit 120 receives the EEG signal amplified by the first amplifier circuit 110 for filtering noises of the EEG signal. The filter circuit 120 is composed of a high-pass filter 122, a low-pass filter 124 and a band reject filter 126. The high-pass filter 122 receives the amplified signal from the first amplifier circuit 110 and removes low frequency drift of the EEG signal so as to prevent interference from low-frequency. The high-pass filter 122 is a Butterworth Filter. In consideration of maintaining the EEG signal as much as possible and simultaneously removes unnecessary high-frequency noises, the low-pass filter 124 is added. The low-pass filter 124 receive the high-frequency part of the EEG signal filtered by the high-pass filter 122 and removes low frequency drift part of the EEG signal so as to prevent interference from high frequency mainly at 60 Hz noise caused mainly by household electrical appliances. Most of the EEG signal falls in the frequency ranging from 1 Hz to 30 Hz so that cut-off frequency is set at 30 Hz. Thus signal at 60 Hz is filtered at once and the low-pass filter 124 works as pre-filter for filtering signal at 60 Hz. The low-pass filter 124 is a Butterworth fourth-order low-pass filter. The band reject filter 126 filters power noise at 60 Hz of the EEG signal being filtered by the low-pass filter 124. The second amplifier circuit 130 receives the EEG signal filtered by the filter circuit 120 and amplifies the filtered EEG signal.

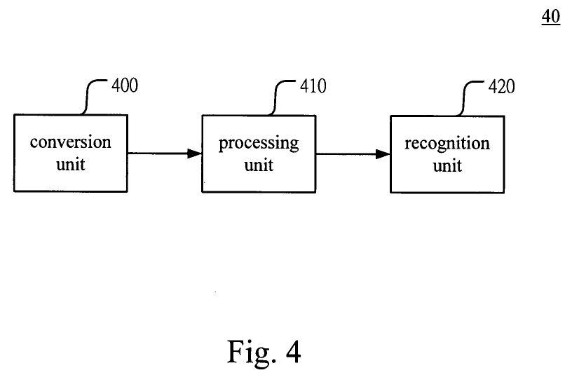

Refer to FIG. 4, it is a block diagram of the processing circuit. As shown in figure, the brain wave signal looks like noise signal and it's dynamic, random, non-periodic and non-linear so that it's difficult to be observed directly. It is learned from previous studies that there are four main frequency hands in the brain wave and different characteristic frequencies are shown in different brain areas and under different drowsiness state. Thus the brain wave is analyzed by a time/frequency domain way. The most common way to analyze time/frequency domain is short time Fourier transform (STFT) which a Fourier-related transform used to determine the sinusoidal frequency and phase content of local sections of a signal as it changes over time. The width of the windowing function relates to the how the signal is represented—it determines whether there is good frequency resolution (frequency components close together can be separated) or good time resolution (the time at which frequencies change). A wide window gives better frequency resolution but poor time resolution. A narrower gives good time resolution but poor frequency resolution. The window or the better resolution is selected. Refer to FIG. 4, the processing circuit 40 consists of a conversion unit 400, a processing unit 410 and a recognition unit 420. The conversion unit 400 receives the EEG signal to generate a conversion signal. The conversion unit 400 is a wavelet transform circuit such as a discrete wavelet transform circuit or a stationary wavelet transform (SWT) circuit. The wavelet transform is applied to time/frequency domain analysis and with feature of multi-resolution. Thus the conversion unit 400 converts brain wave signals (EEG) from six channels, outputs three conversion signals, and take three frequency bands near θ, α and β frequency bands as wavelet coefficients. The processing unit 410 receives and processes the conversion signal tot generate a processing signal. It's difficult and important for the processing unit 410 to get eigenvalue that represents signal characters from the EEG signal being converted by the conversion unit 400. The selection of the signal eigenvalue has great influence on the recognition of the recognition unit 420 afterwards. A proper eigenvalue that enables the signal distinguishable from others will dramatically improve recognition efficiency.

Common methods for finding eigenvalue are using time domain analysis and using frequency domain analysis. In this embodiment, the tow method are used at the same time. First of all, the EEG signal are divided into different frequency bands by wavelet transform and then obtain eigenvalue of each frequency bands by time domain analysis. Thus there is no need to consider frequency characteristics while selecting the features. The features selected in this embodiment are integral value and zero crossing. The use of integral value is for getting frequency band energy while the zero crossing is for getting waveforms of the EEG signal. Thus the processing unit 410 processes these three wavelet coefficients to get 36 eigenvalues (6 channels of EEG signal×3 wavelet coefficients×2 eigenvalues) for data input of the recognition unit 420.

The recognition unit 420 receives and recognizes the processing signal to generate a recognition result. The recognition unit 420 is a neural network such as a back propagation neural network (BPN). By receiving 36 eigenvalues from the processing unit 410, the recognition unit 420 detects the drowsiness of the human body-whether the driver becomes drowsy. Because the recognition unit 420 is a neural network, it must be trained by awake training samples and drowsy training samples collected in advance and the perform drowsiness detection.

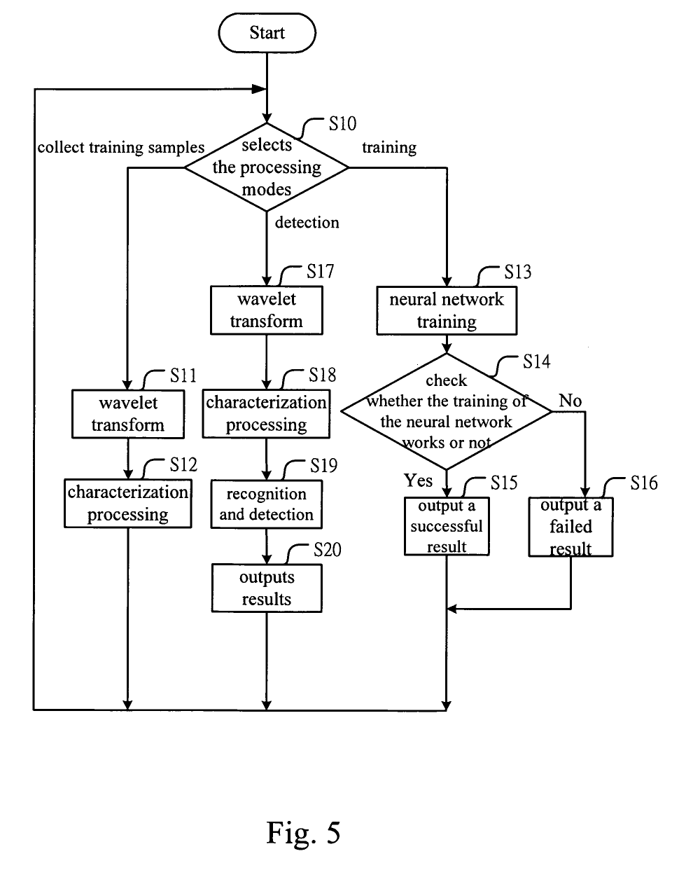

Refer to FIG. 5, a flow chart of the processing circuit is revealed. Before drowsiness detection, the training samples are collected and the neural network needs to be trained. According to the control signal output from the micro-control circuit 30, the processing circuit 40 selects the processing modes, as shown in step S10. Firstly, collect training samples. Refer to the step S11, perform wavelet transform and then run the step S12, perform characterization processing to get the training samples. In this embodiment, awake training samples and drowsy training samples are required. Next tune the step S13, take the neural network training and check whether the training of the neural network works or not, as shown in the step S14. If the answer is yes, output a successful result as shown in the step S15. Otherwise, output a failed result, as shown in the step S16. After finishing the sample collection and the neural network training, perform drowsiness detection. After receiving the EEG signal, the processing circuit 40 performs wavelet transform, as shown in the step S17 and characterizing, as shown in the step S18 so as to obtain a plurality of eigenvalues as input parameters of the neural network. After receiving these eigenvalues, the neural network performs recognition and detection and outputs results to the micro-control circuit 30 for generating a signal to warn the user.

Before the drowsiness detection, sample collection and neural network training need to be performed. Thus the detection system of the present invention further includes an input unit 60 coupled to the micro-control circuit 30 for being input a selection signal to control the micro-control circuit 30. The input unit 60 is formed by buttons. That means a control panel of the input unit 60 is formed by four buttons that users can operate the system easily and conveniently. The functions of each button are respectively: (1): training and retraining of the neural network (2): detection modes (3): starting to get awake training samples (4): starting to get drowsy training samples. Thus the signal generation of the micro-control circuit 30 is under control of the input unit 60 and the processing modes run by the processing circuit 40 is further controlled.

In summary, a drowsiness detection system of the present invention detects a human brain by an EEG detection circuit to generate an EEG signal. Then the EEG signal is sent to a micro-control circuit for generating a control signal. According to the control signal, a processing circuit recognizes the EEG signal to detect drowsiness of the human body.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A drowsiness detection system comprising:
an EEG detection circuit that detects a human brain to generate an EEG signal;
a micro-control circuit that receives the EEG signal and generates a control signal; and
a processing circuit that processes and identifies the EEG for obtaining drowsiness of a body according to the control signal.

2. The system as claimed is claim 1, wherein the processing circuit comprising:
a conversion unit that receives and converts the EEG signal to generate a conversion signal;
a processing unit that receives and processes the conversion signal to generate a processing signal; and
a recognition unit that receives and recognizes the processing signal for generating a detection result so as to learn the drowsiness of the body and the detection result is sent back to the micro-control circuit for output of the detection result.

3. The system as claimed is claim 2, wherein the conversion circuit is a wavelet transform circuit.

4. The system as claimed is claim 3, wherein the wavelet transform circuit is a discrete wavelet transform circuit or a stationary wavelet transform circuit.

5. The system as claimed is claim 2, wherein the processing unit processes the conversion signal is to get integral of the conversion signal.

6. The system as claimed is claim 2, wherein the processing unit processes the conversion signal is to get zero crossings (ZC) of the conversion signal.

7. The system as claimed is claim 2, wherein the system is a neural network.

8. The system as claimed is claim 7, wherein the neural network is a back propagation neural network (BPN).

9. The system as claimed is claim 1, wherein the system further comprising:
an alarm unit coupled to the micro-control circuit and once a detection result shows drowsy state of the body, the alarm unit sends a warning signal.

10. The system as claimed is claim 9, wherein the alarm unit is a light emitting device or an audio device.

11. The system as claimed is claim 10, wherein the light emitting device is a light emitting diode (LED) or a light bulb.

12. The system as claimed is claim 10, wherein the audio device is a speaker.

13. The system as claimed is claim 1, wherein a transmission interface is disposed between the micro-control circuit and the processing circuit for transmission of the control signal.

14. The system as claimed is claim 13, wherein the transmission interface is an enhanced host-port interface (EHPI).

15. The system as claimed is claim 1, wherein the system further comprising:
an analog-to-digital(A/D) conversion circuit receiving the EEG signal and converting the EEG signal into a digital EEG signal.

16. The system as claimed is claim 1, wherein the system further comprising:
an input unit coupled to the micro-control circuit for being input a selection signal to control generation of the control signal by the micro-control circuit.

17. The system as claimed is claim 16, wherein the input unit is a button.

18. The system as claimed is claim 1, wherein the EEG detection circuit comprising:
an electrode module that attaches on the human brain to detect the EEG signal generated from the human brain;
a first amplifier circuit that receives and amplifies the EEG signal;
a filter circuit that receives the EEG signal amplified by the first amplifier circuit and filters the amplified EEG signal; and
a second amplifier circuit that receives the EEG signal filtered by the filter circuit and amplifies the filtered EEG signal.

19. The system as claimed is claim 18, wherein the electrode module comprising six electrodes.

20. The system as claimed is claim 18, wherein the filter circuit comprising:
a high-pass filter that filters low frequency part of the EEG signal;
a low-pass filter that filters high frequency part of the EEG signal filtered by the high-pass filter; and
a band reject filter that filters a noise at certain frequency of the EEG signal filtered by the low-pass filter.

21. The system as claimed is claim 18, wherein the first amplifier circuit is an instrumentation amplifier.

22. The system as claimed is claim 20, wherein the low-pass filter is a Butterworth Filter.
